(19) United States
(12) Patent Application Publication
(10) Pub. No.: US 2012/0020736 A1
(43) Pub. Date: Jan. 26, 2012

(54) METHOD FOR THE RECOVERY OF DEGRADED AREAS USING GENETICALLY MODIFIED PLANT SPECIES

(76) Inventor: Juan Pedro Navarro Avino, Valencia (ES)

(21) Appl. No.: 13/260,980

(22) PCT Filed: Mar. 30, 2009
(86) PCT No.: PCT/ES2009/070080

§ 371 Date: Sep. 29, 2011

(51) Int. Cl. B09C 1/08; A01H 5/00

(52) U.S. Cl. 405/128.7; 800/298; 800/317.3

(57) ABSTRACT

The present invention relates to the search for and choice of forest plants adapted to severe edaphological conditions in soils contaminated by pollutants, which plants are capable of surviving in most parts of the world and cannot enter the trophic chain. The genetic transformation thereof considerably enhances the metal absorption rate and storage capacity thereof and also makes it easier for said absorption to cover the majority of pollutants or harmful substances.

### CROSS REFERENCE TO RELATED APPLICATION

This application is a national stage entry of PCT/ES2009/070080 filed Mar. 30, 2009, under the International Convention.

### TECHNICAL FIELD OF THE INVENTION

The present invention relates to the search and selection of wild vegetal species adapted to severe edaphologic conditions in soils polluted by pollutants, which plants are capable of surviving in most parts of the world and cannot enter the trophic chain. The genetic transformation thereof considerably enhances their metal absorption rate and capacity and also makes it easier for said absorption to cover the majority of pollutants or harmful substances.

The method of said transformations belongs to the field of biotechnology and may be defined as the set of techniques modifying live organisms (or parts thereof), transforming substances of organic origin or using biological processes to bring forth new knowledge, develop products and services.

### BACKGROUND OF THE INVENTION

The industrial revolution birthed very pernicious consequences to the environment due to accumulation of pollutants in the soil, water and the atmosphere.

The soil, being the most stable of these media, allows for longer permanence of pollutants which cannot be degraded during a very long period of time and thus, generate a progressive accumulation, provoking in the first place biodiversity diminishment and initial absence of vegetation, also transferring these elements to other media as air and water, polluting surface as well as underground waters, thus entering in the food chain.

Nevertheless, we observe the existence of a wide variety of vegetal species adapted to these circumstances through a genetic transformation process along time, colonizing even these polluted soils.

These vegetal species known as metalophites have suffered genetic transformations to be able to live in these soils.

Due to their specialization to be able to live in these polluted environments, with specific minerals, in specific areas and edaphic conditions, makes the survival of these species very difficult in other places and if the climate is added to these conditions, there is an increased difficulty for their development in other latitudes.

However, the use of vegetal species to eliminate or accumulate environmentally harmful pollutants is known as phytoremediation, defined as the use of vegetal species to carry on pollutant elimination or transformation actions.

Techniques employed for decontamination of soils basically consist in soil isolation or decontamination.

Isolation techniques avoid propagation of pollution based on the loading of pollutants in appropriate sewers, sealing them in situ or destroying them.

Decontamination and thus soil recuperation techniques pertain to:

The extraction of pollutants through action of a fluid; be it by air (dragging) or water (washing away).

[symbol]

Once dragged the pollutants are cleansed

These are all expensive and inefficient methods

Chemical treatment, that is to say, cleansing the soil through pollutant degrading by chemical reactions, normally oxidation or de-chlorination. Employed in oil products stabilization.

[symbol]

Expensive, complicated and very selective techniques ending up in more soil degradation, un-fertilizing them.

Electrochemical treatment consisting in the displacement of pollutants creating electrical fields, benefiting this displacement by adding water.

[symbol]

It owes to a migration of pollutants phenomena in ionic form through the electrical field.

Electro-osmosis, through movement of liquid in relation to solid surfaces of the electrical field.

Electrophoresis, consisting in the displacement of charged colloidal particles in suspension

These are all very expensive and ineffective procedures.

Thermal treatment, degrading pollutants through heat conveyance.

[symbol]

It is an ex situ treatment with no efficacy for metals.

These treatments leave the soil totally transformed, with no organic matter, without micro-organisms and without any type of biodiversity, rendering said treatments totally inappropriate, besides being, all of them, very expensive.

Microbiological treatment employing certain micro-organisms having degradation capacity (Bio-remediation).

[symbol]

Decontamination by this method is employed in organic pollutants aerobically degraded, though other organic pollutants exist as aliphatic chlorinated ones that must be degraded anaerobically.

This treatment besides being practically only for organic pollutants, needs continuous vigilance so that micro-organisms multiply themselves without loosing their strength; a constant elimination of old micro-organisms having lost degrading power thus enabling them to develop into invasive/mutant species, is also necessary. Besides the aforementioned facts, temperature conditions, pH, micro-organisms strength, etc, also need vigilance.

All of these aforementioned procedures are very expensive and of dubious efficacy.

Phytoremediation, as previously defined, is the technique that employs vegetal species to eliminate pollutants.

[symbol]

Vegetal species employed in phytoremediation have a very selective character, that is to say, they only accumulate one or two metals and they exhibit very low biomass, thus granting them low loading capacity. They grow in very specific areas and possess very short roots, whereby they absorb these metals, whereas their absorption is very superficial.

U.S. Pat. No. 5,364,551—Phytoremediation of Metals

Relates to a process to eliminate ions of metals and describes methods to carry out this purpose.

The method consisting in the extraction of a quantity of metal from a polluted soil containing heavy metals, thus employing transformed members with the adequate vector containing a cDNA codifying sequence for metalothioneine. As already known, it is a protein showing great affinity for divalent heavy metals, such as lead and chrome, thus being claimed in claim 8, that is to say, this patent is selective of said two metals.

Vegetal species with higher absorption capacity, known as hyper-accumulating were discovered afterwards.

Patent WO 0028093—Recovering Metals from Soil

This patent relates to the recovery of metals, such as nickel and cobalt by phytoremediation or phytoextraction from soils rich in metals, where the desired metal is selectively accumulated in hyper-accumulating vegetal species by adjusting the soil pH.

Metals are finally extracted from the tissues of aerial parts of the vegetal species.

But phytoremediation is still slow since these species develop very small biomass and thus, small loading capacity. Besides, they possess a short life cycle, circumstances rendering soil phytoremediation doubtful.

The plants claimed by said patent belong to the Alyssum family.

The main problem with these vegetable hyper-accumulating species, in spite of their high relative metal content, is that they generate small biomass, and thus having small total absorption capacity and thereby the amount of metal extracted is small. Besides, they have a very short life cycle and only grow in much delimited areas.

The unsolved problem to date is the elimination of pollutants with efficacy, that is, to accomplish lower limits than those set by the European Economic Community allowing time periods from one to two years instead of time periods greater than 150/200 years, which is the actual performance of the so called hyper-accumulating plants, that is, a hundred fold decrease in time would render the best solution to eliminate pollutants from the soil.

The present invention solves this problem through search and selection of wild vegetal species adapted to severe edaphological conditions in polluted soils, that is, wild species that have already suffered a natural genetic transformation and have adapted to these conditions, and among these species, those that do not having the possibility to enter the food chain.

Another required condition is its ability to adapt their growth to a great climatic diversity in order to procure a vegetable species able to grown in different climatic conditions. This method has also extended to very wet soils, selecting in this case an arboreal species.

Afterwards a genetic transformation has been accomplished to considerably increase the loading capacity of pollutants and the absorption rate of said elements.

The elements or mixture of same that can be eliminated have been classified in two main groups: noxious and non noxious. Among noxious, by these vegetal species heavy metals as lead, cadmium, mercury, silver, boron, aluminium, iron, manganese, copper, nickel and chromium can be eliminated. Radioactive elements as uranium, rhodium, thorium and plutonium and non noxious elements as sodium, magnesium, lithium, potassium, calcium can also be eliminated.

### BRIEF EXPLANATION OF THE FIGURES

FIG. 1. Represents measurements of the different soil characteristics, for polluted soils type M₄, M₁₅, M₃ and limits for agricultural soil required by the European Union.

Units of these measurements are indicated in the left hand side column. n.d. means: not determined.

FIG. 2. Shows a bar diagram representing growth height (mt) of plants after six months.

The ordinate axis indicates the length in meters of Nicotiana glauca wild plants (wt) after said time and the ones genetically modified with the TaPCS1 OMG gene.

It is confirmed that wild plants grow some three and a half meters high while the genetically modified ones reach up to five meters high. It is to be assumed that in six months time the plants genetically modified have grown a more than 40% in comparison with the wild ones.

FIG. 3. The effect of polluted soils M₄ and M₁₅ on biomass production expressed in grams of total biomass (T), aerial biomass (A), stems and leafs and radicular biomass concentration (R) in mg/kg in Populus tremula×tremuloides cv. Etropole is shown in the figure.

Total accumulated of these following two results expressed in micrograms (μg) are shown: concentrations in stems and leafs (BCF), and concentration in roots (RCF), for wild plants (wt) and genetically modified ones.

FIG. 4. Increase in biomass in Populus tremula×tremuloides cv. Etropole is shown in figure. This figure represents two lines of introduced gene TaPCS1, and two of gene AtPCS1, vs. biomass of a wild plant (the one at the right of the figure), all in M₄ medium, that is to say in a very polluted soil.

It is observed that plants containing anyone of the two genes suffer less the presence of metal in the soil thereby growing more.

FIG. 5. A bar diagram representing in numbers, development of plants Nicotiana glauca (wt) not modified, and those modified with the YCFgene at 26 days.

It is observed that in all cases genetically modified plants with the YCF gene develop more foliage than wild ones.

FIG. 6. A bar diagram representing lengths accomplished by roots in centimetres after 21 days of Nicotiana glauca wild plants (wt) in comparison to those genetically modified with the YCF gene.

It is observed that in all cases root length of modified plants is larger than in wild plants.

### DESCRIPTION OF THE INVENTION

Method for the recovery of degraded spaces using vegetal species genetically modified consists in a series of steps.

It pertains in the first place, to the study (to make a selection complying with a series of requisites) of vegetal species having the capacity to adapt in climatic and edaphological terms.

To that end a series of polluted soils have been defined and classified.

It is to be understood that polluted soil is such whose physical, chemical or biological characteristics have been negatively altered due to the presence of harmful components of human origin, in such concentrations as to impose a risk to human health or to the environment.

A series of samples of polluted soils in mining, industrial and fluvial areas have been taken.

Afterwards, their characteristics have been analyzed from different view points: morphological, food rejection by animals to said vegetal species, environmental and edaphological adaptability, also studying species that would survive in said soils.

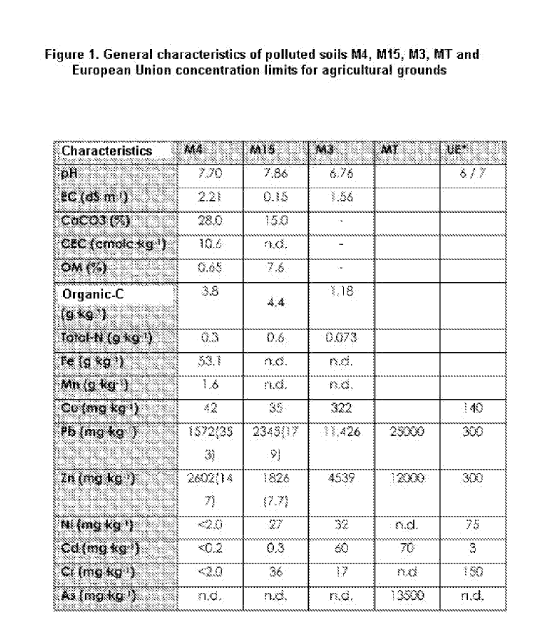

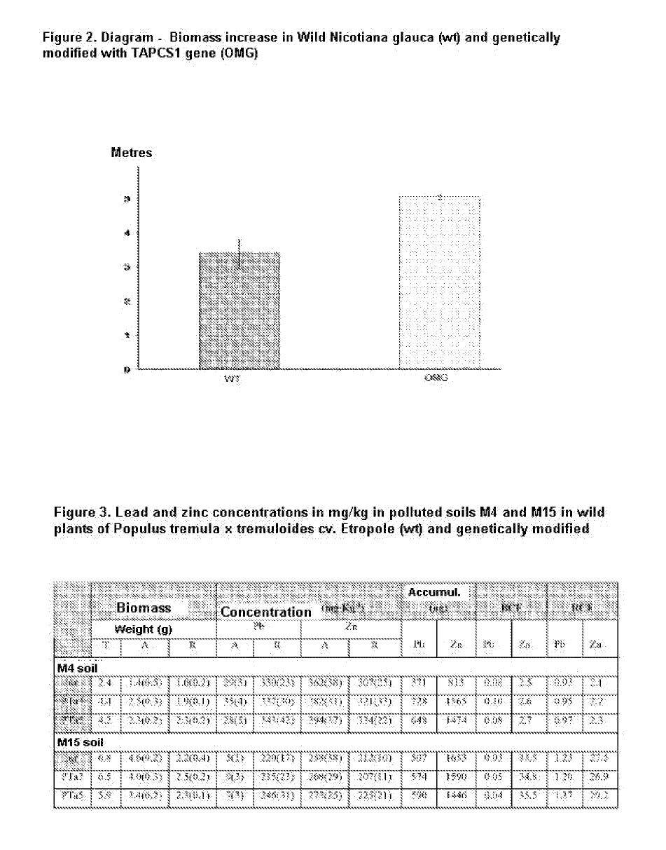

With these data three types of polluted soils have been defined, naming them M₃, M₄, M₁₅ and an MT soil (soil selected from the Turia River bed—Valencia) adjoined in separate table designated as FIG. 1. In said table the quantities of the specified characteristics are indicated in the left hand side column, in units indicated in said column for the three types of soils M₃, M₄, M₁₅. The last column shows the concentration limits established by the European Union for heavy metals in agricultural soils.

Of the species developed in the M₃, M₄, M₁₅ soils and thus adapted to these soils, those that eventually could be part of the trophic chain and those suffering climatic stress upon variation of climatic conditions for a stated time period, have been rejected.

The method went on to the morphological study of vegetal species analyzing their root depth, aspect of great importance in the present method, since it is the organ through which the pollutants are absorbed, considering a profound pursuance of a phytoremediation and not merely a surface one, though acknowledging a slow dissolution of pollutants in the ground.

Afterwards the place or places of the vegetal species where the pollutants extracted from the soil (roots, stems and leafs) were analyzed, since according to their accumulation places they would have a higher or lesser loading capacity of said metals. In FIG. 3 the production of biomass in highly polluted M₄ and M₁₅ soils can be appreciated, as well as lead and zinc concentrations in mg/kg, their totals, expressed in micrograms (μg), concentrations by bio-concentration (BCF) and radicular concentrations (RCF) in wild (wt) and genetically modified (PTa3 and PTa5) vegetal species of Populus tremula×tremuloides cv. Etropole.

Another determining characteristic controlled in this method is the amount of biomass produced by these vegetal species, since its increase generates an increase in loading capacity and thus phytoremediation.

Lastly, vegetal species had to be selected not only having a very easy reproduction but also complying with the requisite of a very abundant reproduction that is, of easy multiplication.

With these criteria vegetal species are selected by exclusion, resulting as best option Nicotiana glauca for dry grounds and Populus tremula×tremuloides cv Etropople for wet grounds.

Moreover the wild Nicotiana glauca (wt) selected has a series of characteristics making it ideal: Could be finally employed as fuel, since It germinates in open ground and its germinating power is very good. It reproduces by cuttings.

When cutting a branch or part of the plant, the plant regenerates that part and goes on growing.

It withstands high ground temperatures and also quite low ones.

It withstands drought and salinity.

It is herbaceous in the first development stages allowing same to have a broad planting frame.

It lignifies soon allowing same to be of good combustion and thus produce caloric and/or electrical energy.

It is seldom or not attacked at all by parasites or diseases favouring stable production efficiency.

It needs very small watering.

Genes TaPCS1 and TaPCS1-AtPCS1 have been respectively introduced in these vegetal species (Nicotiana glauca and Populus tremula×tremuloides).

The selected method was continued by studying the behaviour of vegetal species in their planting and growth, different samples were taken to study the development of same in non polluted control soil (Mₒ) and in polluted soils (M₃, M₄, M₁₅ and MT).

It was observed that the biomass of the selected vegetal species in all types of soils increased in both cases in more than 40% due to their genetic modification with TaPCS1 and AtPCS1 genes.

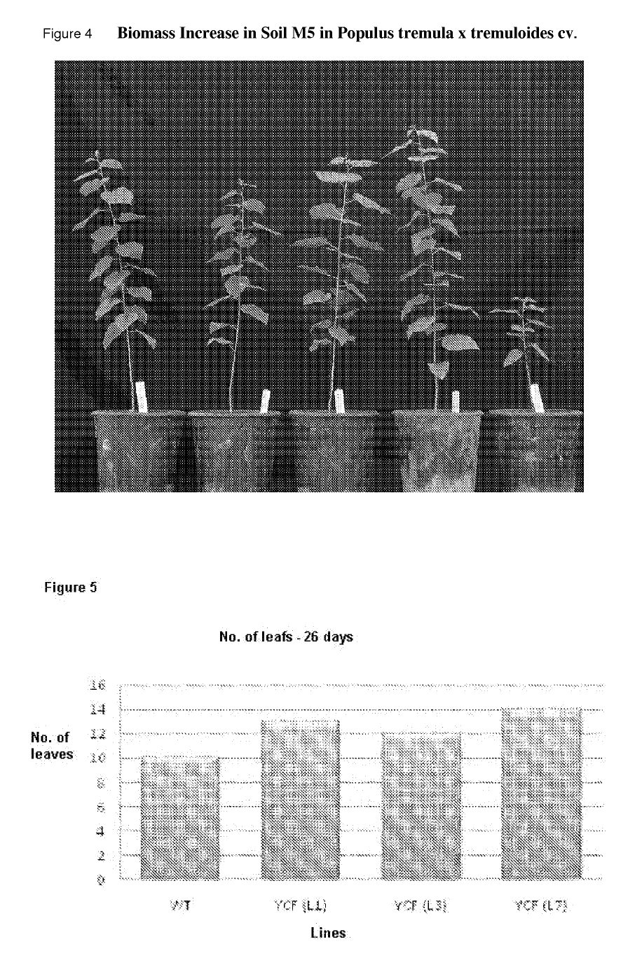

FIG. 4 represents two lines of TaPCS1 and two of ATPCS1 of genes introduced in Populus tremula×tremuloides cv. Etropole, in comparison with the wild plant (the one at the right hand side of FIG. 4), in polluted ground M₄.

These experiments were also carried out in non polluted soils, confirming in all soils the same result: an increase of biomass of modified species, thus constituting a true novelty, that is, the introduction of said genes in a vegetal species increases biomass production in polluted as well as in non polluted soils.

One of the most important characteristics in phytoremediation techniques is the amount of biomass developed by selected vegetal species, even though increase of biomass was surprising upon introduction of TaPCS1 and AtPCS1 genes, the increase of this characteristic with other genes has been investigated revealing that through introduction of the YCF gene, the production of biomass increased in more than 30%, wherefore adding this transformation to those previously obtained with the introduction of the TaPCS1 and AtPCS1 genes, a very important total increase of biomass of plants would be accomplished, besides a considerable time shortening in phytoremediation.

A comparative study of growth in Nicotiana glauca plants genetically modified (GMOs) and not modified was carried out. To that end, the following lines of plants were set out:

wt

L1, L7 and L3 transformed with YCF1 gene.

A study on growth of each of the lines was carried out firstly evaluating the number of leafs in each plant and in a second experiment, the length of roots.

From FIG. 5 the number of leafs developed by non modified plants (wt), and modified with this gene at 26 days can be observed.

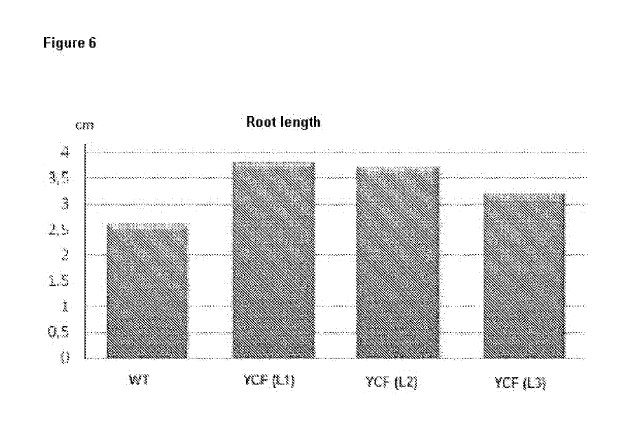

In plants transformed with the YCF1 gene homogenous growth values are observed within the lines and also, superior to the values of wt plants in practically all cases, as may be observed in FIG. 6 regarding he length of roots at 21 days.

Within each group of lines a homogenous radicular growth is observed, since there are no big differences in the length of roots of lines in the same group.

The plants transformed with the YCF1 gene are the ones presenting greater radicular growth, the length of their roots being superior to that of wt plants.

In studying the 3 lines results altogether it is observed that they show a common growth pattern, that is, in the 3 experiments it can be appreciated that the lines transformed with the YCF1 gene provide higher growth values.

As a conclusion, for modified vegetal species the time needed to decontaminate the soil decreases from 100 to 200 fold.

The present methodology employed to introduce the genes through which the increase in the synthesis of phytochelatines is obtained, is as follows:

First, the genes in the adequate plasmid for the vegetal species were included.

In case of the Nicotiana glauca vegetal species the yeast plasmid pYESTaPCS1 containing the phytochelatine synthase gene of Triticum aestivum (TaPCS1) was used. The cDNA of the previously cloned gene in yeast was designated as pYESTaPS1 plasmid.

The plasmid is digested in only one linear cut with XHo I and said cut turned into blunt extremes with the help of the DNA polymerase I. After the change to blunt extremes, the rest of the pYESTaPCS1 plasmid is directed with BamHI to produce a fragment of 2 Kb containing the cDNA of TaPCS1 gene and with extreme 5′ BamHI and 3′ blunt.

Simultaneously, the pBII21 intact plasmid is digested with BamHI and ECL136 II (leaving extreme 3′ blunt to complement with the 3′ of the insert). The insert of 2 Kb binds the sites BamH I-EcI 136 II of the recently cut plasmid, obtaining the new pBITaPCS1 construction.

The new construction (pBITaPCS1) is electropored in a strain of Agrobacterium tumefaciens, C58C1 Rif^{R} Rif (Van Larebeke et al. 1974). The leaf explants of Nicotiana glauca are infected with A. tumefaciens after two days of culture in organogenic medium NB2510 [salts MS (Murashige and Skoog, 1962) including Gamborg vitamins B5, 3% sucrose, 2,5 SYMBOL 109 \f‘Symbol’\s 12 g mL⁻¹ acetic naphthalene (NAA), 1 SYMBOL 109 \f‘Symbol’\s 12 g mL₁ aminopurina bencil (BA) 0.8% agar in darkness. The explants of adult and young leafs are infected through immersion in culture of Agrobacterium during 10 minutes. After one day of co-culturing the explants are transferred to a selective medium NB2510 containing 100 SYMBOL 109 \f ‘Symbol’\s 12 g mL⁻¹ of kanamicin and carbencilin (350 SYMBOL 109 \f ‘Symbol’\s 12 g mL.). Two months after infection, the plants are individually extracted from the explants and transferred to jars containing 30 ml of the B1 medium (MS salts including Gambog B5 vitamins, 0,3 SYMBOL 109 \f ‘Symbol’\s 12 g mL⁻¹ acetic indol acid of 0,2 SYMBOL 109 \f ‘Symbol’\s 12 g mL⁻¹ NAA, 1% sucrose, 100 SYMBOL 109 \f ‘Symbol’\s 12 g mL⁻¹, 0.7% agar).

Besides the TaPCS1 gene, the YCF1 gene (Yeast Cadmium Factor) of Saccharomyces cerevisiae was also introduced in Nicotiana glauca. It is a vacuole carrier enabling the entrance and accumulation of metals in the vacuole. To the sequence of the cDNA of yeast YCF1 gene (Saccharomyces cerevisiae) previously cloned, the cutting sequence of XbaI was added in the extreme 5′ together with that of the 35s promoter (CaMV-Virus of the cauliflower mosaic), to increase gene expression, and in the 3′ extreme, the sequence of the ‘ocs’ terminator together with the cutting site for Sacl. Simultaneously the intact plasmid pGREEN 0179 is digested with SacI and XbaI. The insert binds the sites Sac I-XbaI of the recently cut plasmid obtaining a new construction named pGYCF1. The transformation method is the same, but in this case with 1 to new pGYCF1 construction.

In case of the vegetal species Populus tremula×tremuloides cv. Etropole, the genes introduced are TaPCS1 and AtPCS1 (phytochelatine synthase of Arabidopsis thaliana). Gene AtPCS1 of phythochelatine synthase of Abrabidopsis thaliana was cloned by PCR in an incomplete ORF (in its extreme 5′) of 1458 nt, to which 44 nt were added to complete the codifying sequence in the extreme 5′, together with the sequence GCTggATccACC containing the cutting place of BAMHI enzyme and ‘kozac’ fragment (CACC) in said extreme to over express the AtPCS1 gene. A restriction sequence for EcoRV was also added to the end of the codifying sequence (extreme 3′) allowing its further insertion in the plasmid. Simultaneously the intact pBII21 plasmid is digested with BamHI and EC1136II (leaving extreme 3′ blunt to complement with the 3′ of insert), extracting the uidA gene in its place. The insert of 1.6 Kb (AtPCS1) binds sites BamHI and Ec1136II of the recently cut plasmid, obtaining a new construction named pBIAtPCS1. The transformation method is the same, but in this case with the two constructions, pBIAtPSC1 and pBITaPCS1.

The problem solved with this method consists in identifying the ideal vegetal species for soil decontamination, solving previously exposed problems as:

Decrease of phytoremediation time in 100 to 150 folds.

[symbol]

Increase in biomass production.

[symbol]

Adaptation to different climatic and edaphologic conditions.

[symbol]

Increase of heavy metals extraction range.

[symbol]

Thus, these vegetal species will have the adapting capacity to different climatic and edaphologic conditions, producing a great amount of biomass and accumulating elements or mixtures thereof previously classified in two big groups: noxious and non noxious. Among noxious, with these vegetal species heavy metals as lead, cadmium, mercury, silver, boron, aluminium, iron, manganese, copper, nickel and chromium can be eliminated. Radioactive elements as uranium, rhodium, thorium and plutonium and non noxious as sodium, magnesium, lithium, potassium, calcium, etc. . . .

Besides, the modified N. glauca species has a pleasant appearance, that is to say.

1-5. (canceled)

6. A method for increasing biomass of vegetal species comprising:
growing in dry and wet soils genetic modified plants;
wherein said genetic modified plants are produce by insertions of TaPCS1 and AtPCS1 genes in a first stage and insertion of a YCF gene in a second further stage.

7. The method for increasing biomass of vegetal species according to claim 6, wherein the genetic modified plants in dry soils is Nicotiana glauca species and is produce by insertion of TaPCS1 gene in a first stage and by insertion of YCF gene in a second stage.

8. The method for increasing biomass of vegetal species according to claim 6, wherein the genetic modified plants in wet soils is Populus tremula×tremuloides cv. Etropole and is produce by insertions of the TaPCS1 and AtPCS1 genes in a first stage and by insertion of YCF gene in a second stage.

9. A rapid phytoremediation method for degraded soils by using the combination of a chelating action of TaPCS1, AtPCS1, YCF1 genes with an increase of biomass of vegetal species according to claim 6 comprising:
eliminating heavy elements as lead, cadmium, mercury, silver, boron, aluminium, iron, manganese, copper, nickel and chromium; radioactive elements as uranium, rhodium, thorium and plutonium; and alkaline and earth-alkaline elements by Nicotiana glauca and Populus tremula×tremuloides cv. Etropole, modified by insertions of TaPCS1 and AtPCS1 genes in a first stage, and insertion of YCF gene in a second stage.
